(19) United States
(12) Patent Application Publication
(10) Pub. No.: US 2009/0062805 A1
(43) Pub. Date: Mar. 5, 2009

(54) RONGEUR

(75) Inventor: Guido Casutt, Sulz-Rickenbach (CH)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER GMBH, Winterthur (CH)

(21) Appl. No.: 12/182,778

(22) Filed: Jul. 30, 2008

Related U.S. Application Data
(63) Continuation of application No. PCT/EP2007/050725, filed on Jan. 25, 2007.

(30) Foreign Application Priority Data
Jan. 30, 2006 (EP) 00164.06

(51) Int. Cl. A61B 17/00; A61B 17/32

(52) U.S. Cl. 606/83; 606/170

(57) ABSTRACT

A rongeur (40) has a stem (b), comprising a manipulating end (A) and a working end, and at the manipulating end a grip (42, 44) that is angled in a direction away from the stem. Arranged at a second, working end is a mouth (50), which lies substantially on the side of the stem away from the grip, and a pivotable mouth part (52). The mouth part is mounted at its distal end on the stem in such a way that the opening of the mouth faces the working end of the stem; it is therefore a rongeur of which the working direction, that is to say the scraping and cutting direction of the mouth part when it closes, faces the surgeon. In this case, the stem has a curvature or angling that lies in particular close to the working end, but proximal in relation to the mouth, and which likewise has at least one component pointing away from the angling direction of the grip.

This application is a continuation of PCT Application No. PCT/EP2007/050725, filed on Jan. 25, 2007, entitled RONGEUR, which claims priority to European Patent Application No. 00164.06, filed on Jan. 30, 2006, which are both herein incorporated by reference.

The present invention relates to a rongeur, in particular to a rongeur for nucleotomic applications.

An intervertebral disk operation, also called nucleotomy, can be necessary when a prolapse of the disk results in a combination of failures of cutaneous sensation, muscular strength and/or reflex activity which can be associated with the supply area of a nerve root (radical symptoms).

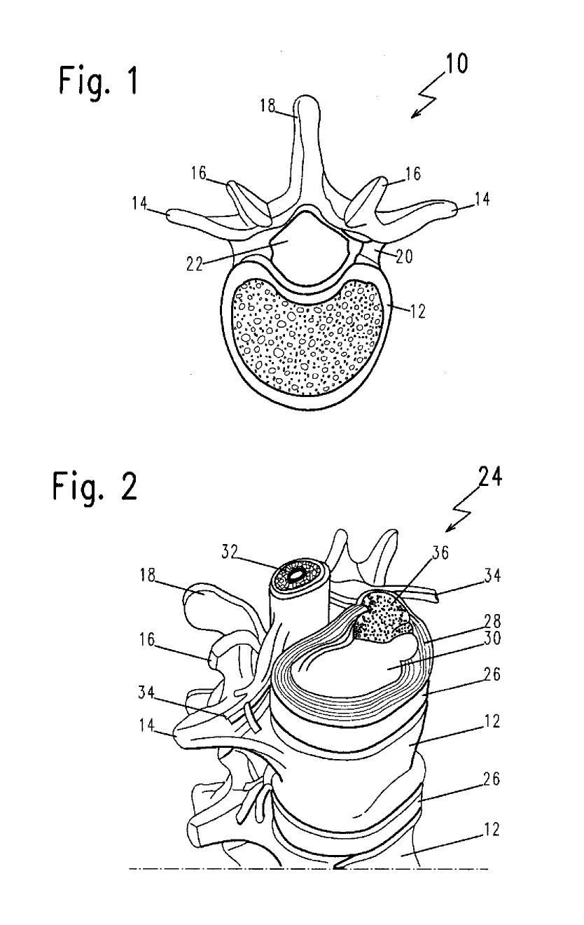

A lumbar vertebra 10, called a vertebra 10 in the following, is shown schematically in FIG. 1. The ventral portion of the vertebra 10 includes a vertebral body 12. Transverse processes 14, joint processes 16 and a spinous process 18 are disposed at dorsal. The spinous process 18 is justified in the sagittal direction, while the transverse processes 14 extend in the lateral direction. A joint process 16 is arranged between a respective transverse process 14 and the spinous process 18. These processes are arranged at the vertebral arch 20 which surrounds the rear of a vertebral foramen 22 in the manner of an arch. The vertebral foramens 22 of the individual vertebrae 10 of the spinal column arranged in rows next to one another form the vertebral canal into which the spinal cord is embedded.

FIG. 2 schematically shows a spinal column portion 24 in a perspective view. The intervertebral disks 26 are arranged between the individual vertebral bodies 12 of the vertebrae 10. The intervertebral disks 26 comprise a fibrous ring 28 which surrounds a soft nucleus pulposus 30. The fibrous ring 28 is made up of concentric collagen fibers and fibrous cartilages. The nucleus pulposus 30 is a cell poor jelly-like tissue with a high water content which acts in a blow damping manner like a water cushion. A portion of a spinal cord 32 is furthermore shown in FIG. 2. Spinal nerves 34 branch off from this between the vertebral arches 20 and the vertebral bodies 12 of the individual vertebra 10 in the planes in which the intervertebral disks 26 are arranged.

Damage to the intervertebral disks can occur, for instance, due to excessive and/or unilateral strains on the spinal column as a result of malpositions or muscular weaknesses or also with a genetic disposition in the different age stages. The intervertebral disks 26 of the spinal column are often subject to progressive degeneration in this respect. Reduced hydration frequently occurs and thus a reduction in the swelling pressure of the nucleus pulposus 30. The fibrous ring 28 is therefore compressed more, with a weakening usually occurring in a posterior region of the intervertebral disk 26. Either the fibrous ring 28 presses against spinal nerves 34 (bulging) or portions of the nucleus pulposus 30 are pressed into the spinal canal (disk herniation, sequestration) due to the collapsing of an intervertebral disk 26 or due to a fissure of the fibrous ring 28. A prolapse, i.e. a slipping down in the marginal region of the intervertebral disk, is shown by way of example and in simplified form in FIG. 2, with a spinal nerve 34 branching off the spinal cord 32 being trapped and irritated by exiting material of the nucleus pulposus 30. Radiating pain, feelings of numbness and possibly paralyses of the musculature supplied by this nerve can occur due to this prolapse of the disk. A large number of patients are affected by such prolapses of the disk.

Such a prolapse can be treated in different ways. The affected vertebral motor segment of the spinal column can thus be stiffened or dynamically stabilized. An artificial intervertebral disk or an artificial nucleus pulposus replacement (nucleoplasty) can also be used. The intervertebral disk 26 is excavated and the nucleus pulposus 30 is removed for the insertion of an artificial nucleus pulposus 30 or of a cage as well as in cases without supply by an implant. The intervertebral disk portions, even the whole intervertebral disk 26 in some cases, pressing onto the nerves are surgically removed (nucleotomy). For this purpose, special bone rongeur forceps, also simply a rongeur, are inserted into the space between two vertebral arches 20 of two vertebrae 10 from dorsal or dorsolateral. The most common surgical methods in this respect represent access from posterior or from posterolateral. In additional a laminectomy or a hemilaminectomy is carried out.

Rongeurs for nucleotomic application are generally known. They include a grip with an elongate, straight shaft attached thereto. A mouth is attached to the end of the shaft disposed opposite the grip and the tissue can be gripped by it in the manner of forceps.

Variants are also known in which the mouth opens on a side of the longitudinal axis of the shaft which is disposed opposite that side at which the grip is arranged. The grip and the mouth opening are therefore arranged so-to-say in mirror fashion with respect to the longitudinal axis of the shaft and the mouth opens in a feed direction. The feed direction describes the direction of an introduction movement of the rongeur into the body of a patient.

Some rongeurs, which are marketed by the Ackermann company (Germany), for example have such a mirrored arrangement of grip and mouth opening. In one variant, the mouth is additionally angled in a direction which is disposed opposite that side at which the grip is arranged. The plane in which the grip extends and the plane in which the mouth opens (mouth plane) are identical in this respect.

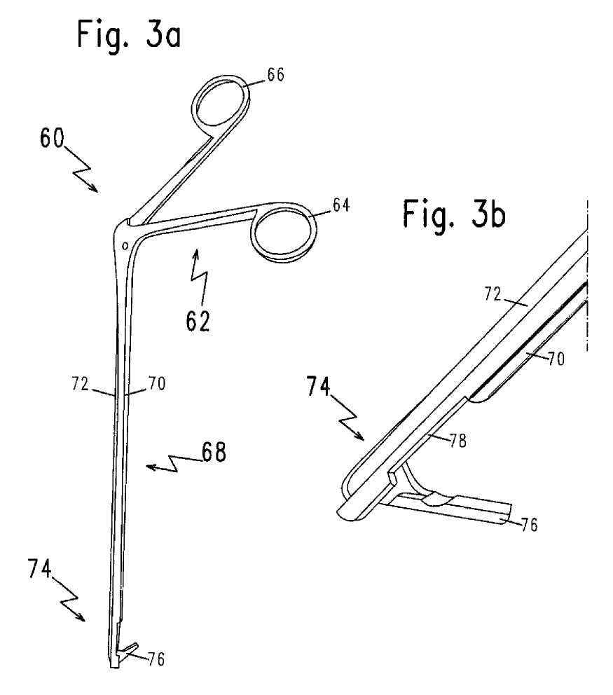

Furthermore, a so-called reverse rongeur is also known which was presented by the company of Teleflex at the Eurospine Congress in Barcelona from Sep. 20 to 24, 2005. This rongeur 60 is shown in FIG. 3a. An elongate, straight shaft 68 having an upper shaft unit 70 and a lower shaft unit 72 adjoins a grip unit 62 which includes a manipulation grip 64 and a grip element 66. A mouth unit 74 with a movable mouth element 76 is arranged at the end of the shaft 68 remote from the grip unit 62.

FIG. 3b shows an enlarged view of the left hand part of the rongeur 60 shown in FIG. 3a. It can be recognized that a mouth element cut-out 78 is associated with the mouth element 76. The mouth element 76 and the mouth element cut-out 78 act together like forceps by actuation of the manipulation grip 64.

In the reverse rongeur in accordance with FIGS. 3a and 3b, the mouth opens—in contrast to a usual pair of craft pliers—in the direction of the grip—that is against the feed direction. Both the grip and the mouth of the rongeur are arranged at a side of the longitudinal axis of the shaft facing the surgeon. In other words, the shaft and the grip of the rongeur substantially form an L geometry an the mouth opens within the region bounded by this L. A U geometry results with a widely opened mouth, represented in simplifying terms.

Further reverse rongeurs are known (website www.endopro.pl) in which the mouth admittedly opens in the same plane as in the previously described known rongeur 60 (FIGS. 3a and 3b).

However, the mouth does not open within the region bounded by the L, but rather on the oppositely disposed side of the shaft. If one were to speak figuratively of an opening of the mouth “downwardly” in the case of the known rongeur 60, the mouth of the further known reverse rongeur would therefore open “upwardly”. These known rongeurs have a straight shaft without a angling or curvature at the working side.

A number of regions of the surgical space can only be reached with difficulty with such rongeurs. It can optionally in particular be necessary to carry out the operation from a plurality of sides in order to ensure that the tissue restricting the nerve has been completely removed. A plurality of surgical accesses may even be necessary for this purpose under certain circumstances.

An innovative rongeur of the initially named kind should now be set forth. The rongeur described in the following, in addition to a series of further properties, simplifies the removal of tissue portions, with in particular the circumstances in a nucleotomic application being taken into account.

The rongeur set forth here includes an end at the manipulation side and an end at the working side, a shaft extending between the end at the manipulation side and the end at the working side, a grip section arranged at the end of the shaft at the manipulation side and angled with respect to the shaft and a mouth arranged at the end of the shaft at the working side. The shaft furthermore includes a first straight shaft section at its end at the manipulation side having a first longitudinal axis and a second straight shaft section at its end at the working side having a second longitudinal axis, with the second longitudinal axis including an angle with respect to the first longitudinal axis such that the second straight shaft section faces into a first half-space starting from a first straight shaft section. The first half-space is geometrically defined as a half-space which is complementary to a second half-space in which the grip section is disposed, with the half-spaces being bounded by a plane in which the first straight shaft section extends and which is perpendicular on a plane defined by the first straight shaft section and the angled grip section, with the mouth being arranged in the second straight shaft section and having a mouth part pivotable around a pivot axis and with the pivot axis being arranged at a distal end of the mouth part such that the pivotable mouth part extends away from the end of the shaft at the working side with a closed mouth such that the mouth substantially faces in the direction of the end of the shaft at the manipulation side in the open state. It is thus a case of a so-called “reverse rongeur” as defined above. The mouth is furthermore arranged on a side of the shaft facing the grip, that is the mouth part opens in a direction facing into the first half-space.

In the following, the first straight shaft section is also called a “shaft” and the second straight shaft section is also called a “mouth section”.

The instrument can in other words be described as follows: The rongeur has a shaft with an end at the manipulation side and an end at the working side and a grip angled in a direction from the shaft at the end at the manipulation side. A mouth is arranged at a second end at the working side which is substantially disposed on the side remote from the direction in which the grip is angled, as well as a pivotable mouth part. The mouth part is supported at its distal end at the shaft so that the opening of the mouth faces toward the end of the shaft at the working side; it is therefore a case of a rongeur whose working direction, that is the scraping and cutting direction, of the mouth part when closing, faces the surgeon. In this respect, the shaft has a curvature or angled portion which is in particular disposed close to the end at the working side, but proximal from the mouth and which likewise has at least one component facing away from the angling direction of the grip. Proximal is to be understood in this respect as “toward the end at the manipulation side” and distal as “toward the end at the working side”.

With the rongeur set forth here, tissue parts can be gripped with the mouth, in a similar manner as with conventional forceps. Unlike with conventional forceps, however, the mouth opens in the direction of the grip section or of the end of the shaft at the manipulation side, that is against the feed direction (reverse rongeur). Unlike some of the known reverse rongeurs mentioned above, the mouth, however, does not open in a half space in which the grip section is disposed and which is bounded by a plane in which the shaft extends and which is perpendicular on a plane defined by the shaft and the grip section (shaft-grip plane). The mouth rather opens in the half space complementary thereto. With this reverse rongeur, it is therefore possible to speak of a Z geometry in the widest sense with an open mouth, with the middle line of the Z being formed by the shaft, which figuratively expresses the arrangement of the grip and mouth mirrored with respect to the longitudinal axis of the rongeur or to the first straight shaft section. In other words, the plane which is formed by the open mouth and in which the pincer movement of the mouth takes place (mouth plane) can be rotated by any desired angle between 90° and 270° without respect to the shaft-grip plane around the longitudinal axis of the grip section (starting from the grip of the grip section). The mouth plane does not have to be identical to the grip-shaft plane.

Furthermore, with the rongeur set forth here, unlike the known reverse rongeurs, the mouth is additionally arranged angled or curved with respect to the shaft, i.e. the rongeur has an angled portion or curvature at the working side. The mouth is therefore not disposed on the longitudinal axis of the shaft.

The end of the shaft at the working side has a curvature-free straight second shaft section or mouth section. The mouth which has a pivotable mouth part is arranged in this mouth section. The mouth is thus not a punch-like element (“puncher”), but is rather similar to forceps. In other words, the mouth includes a foldable jaw with which the regions of the intervertebral space which are masked—from the view of the surgeon—can be reached and efficiently excavated. The pivotable mouth part thus has a scraping function similar to a curette on actuation and, due to its pivot radius, has a specific range within which material is excavated on the activation of the rongeur, which clearly distinguishes the function from that of a puncher.

The rongeur is in particular shaped and dimensioned at the working side such that it is possible with this rigid instrument, on the one hand, to move past the spinous processes 18 on the introduction of the rongeur and, on the other hand, to excavate the total nucleus when the rongeur is introduced. The length should therefore be dimensioned such that, on the one hand, the total intervertebral disk space can be gripped and, on the other hand, the instrument can be handled and maneuvered between the vertebrae in the small available inlet window. The longitudinal extent of the second straight shaft section or of the mouth section amounts, for example to between 15 mm and 45 mm. With a shorter mouth section, the mouth part associated with the mouth section would be less suitable to grip all the regions of the intervertebral disk space. A mouth section with a longitudinal extent of more than 45 mm would, in contrast, not have any further advantages with respect to the accessibility of all regions of the intervertebral disk space and would therefore be unnecessarily bulky and awkward in handling.

The angled portion or curvature of the rongeur at the working side or the angle between the first and second straight shaft sections, for example, includes an angular range from approximately 15° to 35°. With an angle of at least 15°, the mouth section can easily reach the regions of the intervertebral disk space which are covered—from the view of the surgeon. At an angle of more than 35°, the introduction of the rongeur into the body of the patient would prove to be increasingly difficult to impossible since in particular the spinous processes of the vertebra restrict the available manipulation leeway.

The introducibility at a given angle furthermore also depends on the transition geometry between the first and second shaft sections. In an embodiment, a transition region between the first straight shaft section and the second straight shaft section is formed as an arch segment with a substantially constant curvature and indeed such that the transition region substantially represents a section of an arc of a circle. The radius of curvature of the arc segment amounts, for example, to between 50 mm and 125 mm.

Such a defined curvature on the one hand extends such that the rongeur is simple to handle. On the other hand, the angle of curvature defined by the above values is large enough to reach the whole intervertebral disk space with the mouth section. It can be set down as a rule of the thumb that a large radius of curvature makes a large angle possible, but, on the other hand, does not facilitate the handling overall, in particular on access and on the moving out of the instrument.

Due to the geometrical design of the rongeur set forth here, the operation is simplified and all irritating tissue portions, in particular the whole nucleus, can be reliably removed since the regions relevant in an operation are reachable and it is in particular possible to guide the instrument or its end region at the working side around the spinous processes with the mouth section. An ideal control of the surgical instrument and above all of the mouth is of special importance since, in the case of a nucleotomy, the operation take place close to the spinal cord and damage to the spinal cord is to be avoided at all costs in the course of the operation.

In an embodiment, the first straight shaft section and the second straight shaft section are disposed in a plane which is at least substantially identical to a plane in which the opening of the mouth is disposed.

In accordance with a further embodiment, the opening of the mouth is disposed in plane which is at least substantially identical to a plane which is defined by the first straight shaft section and the angled grip section.

In an embodiment, the first straight shaft section and the second straight shaft section are disposed in a plane which is at least substantially identical to a plane which is defined by the first straight shaft section and the angled grip section.

The different embodiments are adapted to the respective prevailing operation conditions, with different combination possibilities of the described geometries being conceivable. The surgeon can therefore select the instrument suitable for the situation to obtain good results and to make the procedure as gentle as possible.

A further embodiment of the rongeur has a depth scale at the mouth and/or at the shaft. These markings serve for the orientation of the surgeon who can thus estimate how deep the surgical instrument is located in the body of the patient. This control of the position of the rongeur simplifies the operation and also represents a safety aspect.

A further orientation aid is realized in a further embodiment. In a region of the shaft facing the grip region, at least one display means is provided that indicates the direction of the curvature or angled section at the working side, and thus the mouth plane, to the surgeon.

Further embodiments are set forth in the dependent claims, in the description and in the drawings.

The invention will be described in the following purely by way of example with reference to possible embodiments and to the drawings.

FIG. 1 shows a schematic representation of a vertebra (see introduction).

FIG. 2 shows a perspective view of a schematically shown spinal column portion obliquely from above (see introduction).

FIG. 3a shows a reverse rongeur such as was presented at the European Congress in September 2005.

FIG. 3b shows an enlargement of the mouth of a reverse rongeur of FIG. 3a.

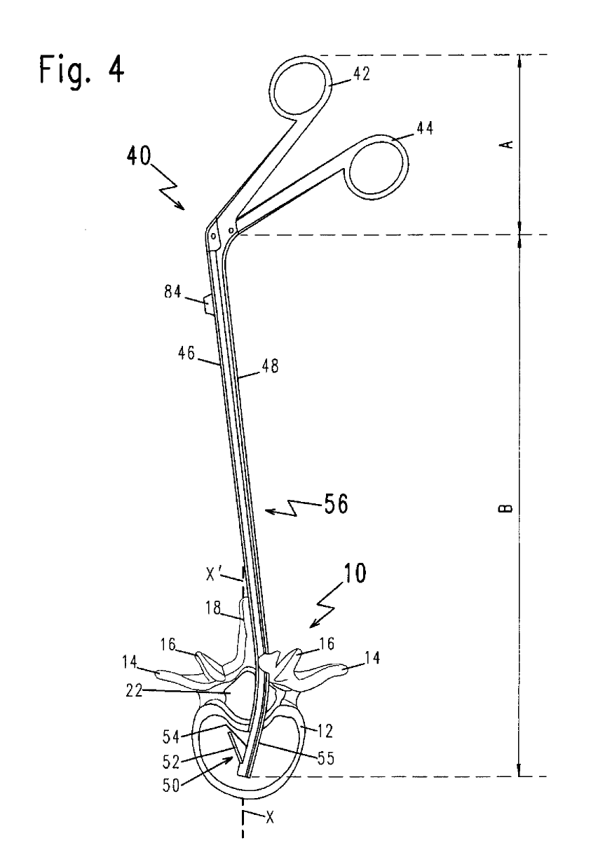

FIG. 4 shows a rongeur which is introduced into the intervertebral disk space from posterior.

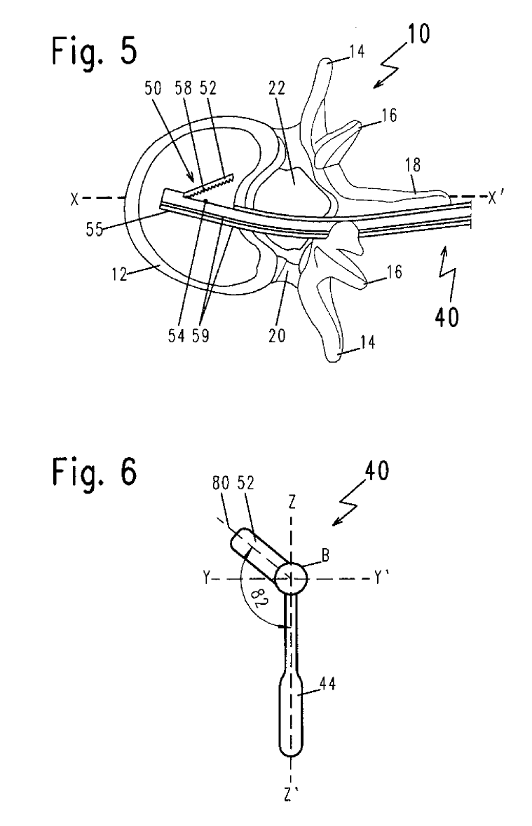

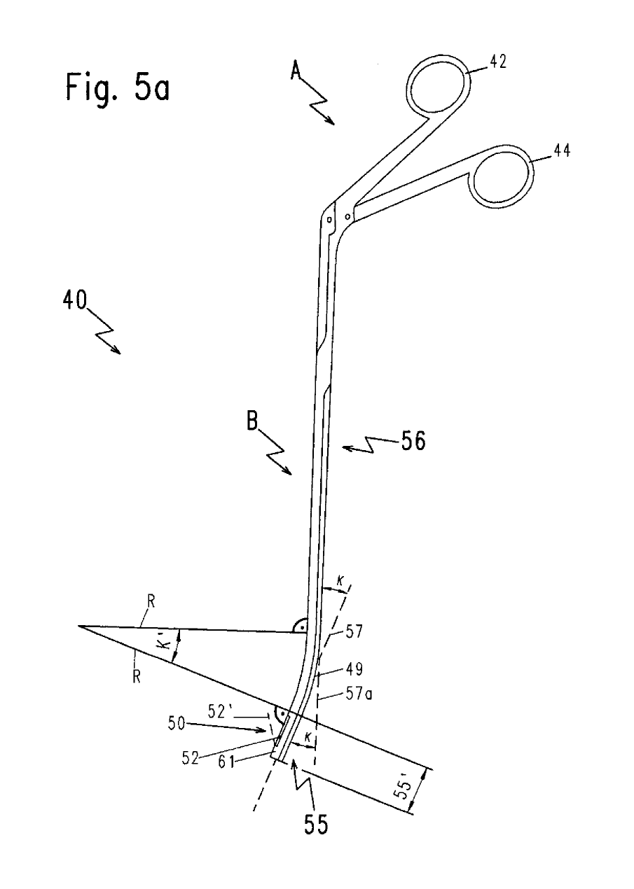

FIG. 5 shows an enlargement of the left hand image section of FIG. 4 with a vertebra and the rongeur end at the working side.

FIG. 5a shows an embodiment of a rongeur.

FIG. 6 schematically shows a rongeur with a straight shaft from the front for the explanation of possible orientations of the mouth.

An embodiment of a rongeur 40 is shown in FIG. 4. It includes a grip section A and a shaft B. The grip section A at the manipulation side has a holding grip 42 and an actuation grip 44. The elongated shaft B with a first straight shaft section 56 has an upper shaft element 46 and a lower shaft element 48. The mouth 50 is located at the end of the rongeur 40 at the working side which includes a second straight shaft section which is also called a mouth section 55 in the following. The mouth 50 includes a mouth part 52 and a mouth cut-out 54 associated with the mouth part 52. The vertebra 10 is moreover shown. In this representation, the rongeur has been introduced into the intervertebral disk space through an opening between the spinous process 18 and the joint process 16. Further details of the end of the rongeur at the working side can be seen from FIG. 5.

FIG. 4 illustrates in a particularly illustrative manner the advantages of the functional elements of grip and mouth attached with respect to the longitudinal axis of the rongeur 40—i.e. with respect to the longitudinal axis of the first straight shaft section 56—in complementary half spaces—or in other words—at different sides. Intervertebral disk material masked from the view of the surgeon by the spinal cord 32 in the vertebral foramen 22 can thereby also be gripped. This is also facilitated by the curved extent of the transition region between the first straight shaft section 56 and the second straight shaft section (mouth section) 55.

A rongeur of the known type would only grip material which is disposed substantially beneath the sagittal plane (indicated by the line XX′) on a surgical procedure through the same opening between the spinous process 18 and the lower joint process 16. Regions of the intervertebral disk space previously not accessible or only accessible with difficulty are reached by the Z geometry of the rongeur 40 and the angled portion of the mouth section 55 with respect to the shaft B. The rongeur 40 above all makes it possible due to its shape also to work around the spinal cord 32 which is located in the vertebral foramen 22. This makes possible a gentle manner of treatment, with it simultaneously being ensured that the tissue to be removed is completely gripped.

Since the vertebrae 10 differ with respect to their size and shape along the vertebral column, different rongeurs 40 can also have different sizes and shapes and can thereby be optimized for the respective task. The relates both to the length of the shaft B or of the first straight shaft section 56, the embodiment of the grip section A and the size and geometry of the mouth 50. The rongeur 40 can also have very different types of and differently pronounced angled portion and curvature in its elongate extent. Embodiments can also be provided with a mouth 50 angled or curved with respect to the shaft B.

In the rongeur 40 shown, the mouth part 52 is moved by a movement of the actuation grip 44. In this respect, the actuation mechanism not shown here and also not explained in more detail can be configured such that the mouth 50 is closed when the actuation grip 44 is actuated, that is when the actuation grip 44 and the holding grip 42 have been moved toward one another. The converse case, that is a closed mouth 50 when the actuation grip 44 is not actuated, can likewise be realized in a simple manner. The movement of the actuation mechanism can be transmitted from the grip section A to the mouth 50 by a relative movement of the upper shaft element 46 with respect to the lower shaft element 48. A plurality of different grips and actuation mechanisms can be realized.

The rongeur 40 is additionally provided with a mouth plane indicator 84. In this only exemplary case, the mouth plane indicator 84 is a small area which is aligned parallel to the mouth plane, i.e. to the plane in which the mouth opens. The mouth plane indicator 84 thereby indicates the location of the mouth plane to the surgeon and thus facilitates the orientation of the instrument in the body of a patent during the operation. Depending on the embodiment, such indicator means can have different designs, e.g. be made as a rod or as in indicator and for example also indicate the location of a plane of curvature/angling of the end of the shaft B at the working side. Such indication means are of special importance with complex geometrical embodiments of the rongeur 40.

FIG. 5 shows an enlarged section of the end of the rongeur 40 at the working side in a position within an intervertebral disk space, as typically occurs in an operation. The mouth 50 is opened the mouth part 52 is provided with a profiling 58 to be able to grip and fix intervertebral disk parts more easily. In the closed state, the mouth part 52 substantially completely fills the mouth part cut-out 54 associated with it. It is thereby achieved that the cross-section of the end of the rongeur 40 at the working side is as small as possible and projecting parts are avoided which could injure healthy tissue. Markings 59 are additionally applied to the rongeur 40. They facilitate the positional determination of the rongeur 40 within the intervertebral disk space. These markings 59 can be engravings, for example Other marking possibilities are, however, also conceivable.

As can be seen from FIG. 4, it can also be seen from FIG. 5, that due to the curved extent of the rongeur 40, it is essentially possible to work around the sensitive spinal cord 32. Injuries to the spinal cord 32 are thus largely avoided. It can equally be recognized that tissue portions which are arranged above the sagittal plane XX′ from the view of the surgeon are reached. A known rongeur 40 with a reverse opening of the mouth 50 would only be able to reach a small portion of the intervertebral disk space beneath the sagittal plane XX′. Without an angled portion in the course of the rongeur 40, a region can in particular be reached which is arranged just behind the spinal cord 32 from the view of the surgeon. The rongeur 40 set forth here therefore makes it possible for a number of nucleotomic procedures to manage only with one operational hole.

FIG. 5a shows a rongeur 40 and illustrates an embodiment of the end at the working side. The end of the rongeur 40 at the working side includes a straight mouth section or a second straight shaft section 55 which does not have any curvature in its course. Its longitudinal extent is therefore substantially defined by a longitudinal mouth section axis 57. As can be seen from FIG. 5a, the longitudinal mouth section axis 57 and a longitudinal axis 57a of the first straight shaft section 56 include an angle K which amounts in this case approximately to 22°. Other angles can be provided as required. It can be seen from simple geometrical considerations that the angle K′ in FIG. 5a corresponds to the angle K. The angle K is in particular in the range from 15° to 35°.

A transition region 49 of the shaft B adjoins the end of the second straight shaft section 55 at the manipulation side and is formed as a curvature shaped like an arc of a circle or as a section of an arc of a circle and which is defined—in addition to the angle K—by the radius of curvature R of this arc segment. The radius R is in particular in the range from 50 mm to 125 mm.

A suitable dimensioning of an extent 55′ of the straight shaft section 55 serves, on the one hand, not to design the rongeur 40 as unnecessarily bulky, but also, on the other hand, to enable an efficient and complete excavation of the intervertebral disk space. The length of the second straight mouth section 55, that is its extent 55′, is in particular in the range from 15 mm to 45 mm.

The mouth part 52 pivotable around a pivot axis 61 disposed in the digital region of the mouth section 55 is shown in FIG. 5a in the closed state. An open mouth part 52′ is indicated by a dashed line. Since the pivotable mouth part 52 extends away from the end of the shaft B at the working side, that is in the direction of the transition region 49 starting from the pivot axis 61, with a closed mouth, the mouth faces substantially in the direction of the end of the shaft B at the manipulation side in the open state.

FIG. 6 schematically shows a rongeur 40 in a frontal view which is shown for reasons of simplicity for the explanation of different mouth orientations with a straight shaft B, that is without an angled mouth section facing into the first half space (upwardly here). In this view, the actuation grip 44 covers the holding grip 42 (not shown) disposed behind it. The mouth 50 is open, i.e. the movable mouth part 52 is located in its outwardly pivoted position. In this respect, the rear side—that is the side remote from the mouth cut-out 54 (not shown)—of the mouth part 52 can be seen. The half-space boundary plane YY′ is in each case disposed perpendicular to the drawing plane and to the shaft-handle plane ZZ′ which is defined by the grip section A. The shaft-grip plane ZZ′ is therefore the drawing plane in FIG. 4, for example.

It becomes clear from FIG. 6 that the mouth 50 opens into a half space which is bounded by the half-space boundary plane YY′ and which is remote from the grip. In other words, the projection of the longitudinal axis of the mouth part 52 onto the drawing plane—the mouth part axis 80—can include a mouth-grip angle 82 of 90° up to 270° with the grip in the shaft-grip plane ZZ′.

Contrary to what is shown in FIG. 6, the rongeur 40 is actually provided with at least one curvature or angled portion at the working side, as was explained above, with this generally being able to have any desired design. Due to the combination of one or more such angled portions/curvatures with any desired mouth-grip angle 82 between 90° and 270°, different complex geometrical shapes of the rongeur 40 can be realized to provide a rongeur 40 which is optimized for specific surgical conditions.

### REFERENCE NUMERAL LIST

10 vertebra
12 vertebral body
14 transverse process
16 joint processes
18 spinous process
20 vertebral arch
22 vertebral foramen
24 vertebral column section
26 intervertebral disk
28 fibrous ring
30 nucleus pulposus
32 spinal cord
34 spinal nerve
36 prolapse
A grip section
B shaft
40 rongeur
42 holding grip
44 actuation grip
46 upper shaft element
48 lower shaft element
49 transition region
50 mouth
52 mouth part
54 mouth cut-out
55 mouth section (second straight shaft section)
55′ extent of the mouth section
56 first straight shaft section
57 longitudinal mouth section axis
57a longitudinal axis of the first shaft section
58 profiling
59 marking
60 rongeur
61 pivot axis of the mouth part
62 grip unit
64 manipulation grip
66 grip element
68 shaft
70 upper shaft unit
72 lower shaft unit
74 mouth unit
76 mouth element
78 mouth element cut-out
80 mouth part axis
82 mouth-grip angle
84 mouth plane indicator
K, K′ curvature angle
R radius
XX′ sagittal plane
YY′ half-space boundary plane
ZZ′ shaft-grip plane

1. A rongeur comprising:
a shaft having a manipulation end and a working end;
a grip disposed at the manipulation end of the shaft; and
a mouth disposed at the working end of the shaft;
wherein the shaft includes first and second straight shaft sections and a curved section therebetween, the curved section extending in a direction away from the grip, wherein the mouth is disposed on the second straight section.

2. The rongeur of claim 1, wherein the shaft includes an upper shaft element and a lower shaft element.

3. The rongeur of claim 1, wherein the mouth includes a mouth part pivotable around a pivot axis disposed at a distal end of the mouth part such that the pivotable mouth part extends away from the working end of the shaft in an open position with the open mouth facing the manipulation end of the shaft.

4. The rongeur of claim 3, wherein the first straight shaft section and the grip define a first plane, wherein a second plane is perpendicular to the first plane and defines a first space extending to a first side of the second plane and a second space extending to a second side of the second plane, wherein the second straight shaft section extends into the first space and the grip extends into the second space.

5. The rongeur of claim 4, wherein the mouth part in the open position extends from the second straight shaft section within the first space, and an angle between the open mouth part and the grip is from about 90 degrees to about 270 degrees.

6. The rongeur of claim 1, wherein the first straight section of the shaft has a first longitudinal axis and the second straight section of the shaft has a second longitudinal axis, wherein the second longitudinal axis includes an angle with respect to the first longitudinal axis, wherein the angle is from about 15 to about 35 degrees.

7. The rongeur of claim 1, wherein the second straight shaft section has a length of from about 15 mm to about 45 mm.

8. The rongeur of claim 1, wherein the curved section is an arcuate segment with a substantially constant curvature such that the curved section substantially represents a section of an arc of a circle.

9. The rongeur of claim 8, wherein a radius of curvature of the section of an arc of a circle is between about 50 mm and about 125 mm long.

10. The rongeur of claim 4, wherein an opening of the mouth, the first straight shaft section, and the grip are disposed in a plane which is substantially identical to the second plane.

11. The rongeur of claim 4, wherein the first straight shaft section and the second straight shaft section are disposed in a plane which is at least substantially identical to the second plane.

12. The rongeur of claim 1, further including depth markings.

13. The rongeur of claim 1, further including at least one indicator provided on the first straight section of the shaft, wherein the indicator indicates a plane in which the mouth opens.

14. A method of cutting bone or cartilage comprising:
providing a rongeur, the rongeur having a shaft having a manipulation end and a working end, a grip disposed at the manipulation end of the shaft, and a mouth disposed at the working end of the shaft, wherein the shaft includes first and second straight shaft sections and a curved section therebetween, the curved section extending in a direction away from the grip, wherein the mouth is disposed on the second straight section;
positioning the rongeur such that the curved section faces the site where bone or cartilage is to be cut; and
actuating the grip thereby actuating the mouth to cut the bone or cartilage.

15. The method of claim 14, wherein the bone or cartilage to be cut includes intervertebral disc material, wherein the step of positioning the rongeur includes inserting the rongeur between two adjacent vertebra from a posterior position.

16. The method of claim 15, wherein the step of inserting the rongeur includes inserting the rongeur between a spinous process and a joint process.

17. The method of claim 15, wherein the step of inserting the rongeur includes inserting the shaft of the rongeur substantially along a first side of a sagittal plane with the grip and first straight shaft section on the first side of the sagittal plane and the curved section and mouth extending across the sagittal plane to a second side of the sagittal plane.

18. The method of claim 17, further comprising actuating the mouth of the rongeur and moving the rongeur to substantially remove the entire intervertebral disc through a single incision.

19. A kit comprising a plurality of rongeurs, each rongeur having a shaft having a manipulation end and a working end, a grip disposed at the manipulation end of the shaft, and a mouth disposed at the working end of the shaft, wherein the shaft includes first and second straight shaft sections and an angled section therebetween, the angled section extending in a direction away from the grip, wherein the mouth is disposed on the second straight section; wherein the plurality of rongeurs differ in at least one of first straight shaft section length, second straight shaft section length, angle of the angled section, size of the mouth, and geometry of the mouth.
